(19) United States
(12) Patent Application Publication
(10) Pub. No.: US 2026/0062371 A1
(43) Pub. Date: Mar. 5, 2026

(54) A METHOD AND A SYSTEM FOR PRODUCING ETHER

(71) Applicant: Neste Oyj, Espoo (FI)

(72) Inventors: Antti PYHÄLAHTI, Porvoo (FI); Matti KOSKINEN, Porvoo (FI); Antti KURKIJÄRVI, Porvoo (FI); Hannu NOUSIAINEN, Porvoo (FI); Hanna-Maija HONKANEN, Porvoo (FI)

(73) Assignee: Neste Oyj, Espoo (FI)

(21) Appl. No.: 19/106,587

(22) PCT Filed: Aug. 7, 2023
(86) PCT No.: PCT/FI2023/050456

§ 371 Date: Feb. 25, 2025

(30) Foreign Application Priority Data
Aug. 26, 2022 (FI) 20225755

(51) Int. Cl. C07C 41/06; B01D 3/14; B01J 8/04; B01J 8/06; B01J 8/18; B01J 8/26; C07C 41/42

(52) U.S. Cl. CPC C07C 41/06; B01D 3/143; B01J 8/0492; B01J 8/0496; B01J 8/065; B01J 8/067; B01J 8/1827; B01J 8/1836; B01J 8/26; C07C 41/42; B01J 2208/00176; B01J 2208/065

(57) ABSTRACT

A system for producing ether includes a processing system that receives alcohol and olefinic hydrocarbon feedstock and includes a first etherification reactor system that produces a first reaction effluent. A distillation column system receives a mixture containing the first reaction effluent to produce ether. A side flow outlet withdraws a side flow from the distillation column system and a second etherification reactor system receives the side flow and produces a second reaction effluent. The second reaction effluent is mixed with the first reaction effluent, and this mixture is supplied to the distillation column system. The side flow makes it possible to produce a given amount of ether with a smaller amount of catalyst material.

### FIELD OF THE DISCLOSURE

The disclosure relates generally to producing ethers such as tertiary alkyl ethers which can be used for example as components of motor fuels. The produced ethers may contain for example methyl t-butyl ether, ethyl t-butyl ether, t-amyl methyl ether, t-amyl ethyl ether, or mixtures thereof, and possibly heavier tertiary alkyl ethers. More particularly, the disclosure relates to a method for producing ethers and to a system for producing ethers.

### BACKGROUND

Tertiary alkyl ethers can be used as additives of gasoline to improve anti-knocking characteristics and to reduce harmful components in exhaust gases. The oxygen-containing ether group of tertiary alkyl ethers has been found advantageous to improve a combustion process of automotive engines and other similar engines. Examples of suitable tertiary alkyl ethers are: methyl t-butyl ether “MTBE”, ethyl t-butyl ether “ETBE”, t-amyl methyl ether “TAME”, t-amyl ethyl ether “TAEE”, t-hexyl methyl ether “THME”, and t-hexyl ethyl ether “THEE”. These ethers can be produced by etherification of olefinic hydrocarbon feedstock containing tertiary iso-olefins with monovalent aliphatic alcohols i.e. alkanols. The etherification reactions can be carried out in a fixed bed reactor, in a fluidized bed reactor, in an ebullated bed reactor, in a boiling-point reactor, in a tubular reactor, or in a catalytic distillation column.

A typical system for producing ethers, such as tertiary alkyl ethers, comprises one or more etherification reactors and a distillation column configured to receive a reaction effluent produced by the one or more etherification reactors. The produced ether is typically taken out from the bottom of the distillation column. The etherification reactions in the one or more etherification reactors take place in the presence of catalyst material which may comprise for example cation exchange resin or zeolite. The catalyst material within each etherification reactor gets gradually deactivated during the etherification reactions, and thus there is a need to replace the deactivated catalyst material with active catalyst material. The replacement of the deactivated catalyst material with the active catalyst material causes material costs due to the price of the catalyst material as well as labor costs due to the work needed for replacing the deactivated catalyst material. Furthermore, the time needed for replacing the deactivated catalyst material may cause downtime costs depending on a system for producing ethers. Therefore, there is a need for technologies to reduce the amount of catalyst material needed in ether production.

### SUMMARY

The following presents a simplified summary to provide a basic understanding of some embodiments of the invention. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

In accordance with the invention, there is provided a new method for producing ether such as tertiary alkyl ether which may comprise for example methyl t-butyl ether “MTBE”, ethyl t-butyl ether “ETBE”, t-amyl methyl ether “TAME”, t-amyl ethyl ether “TAEE”, t-hexyl methyl ether “THME”, t-hexyl ethyl ether “THEE”, and/or mixtures thereof.

A method according to the invention comprises:
supplying alcohol and olefinic hydrocarbon feedstock, for example C₄₋₇ olefins, to a processing system comprising a first etherification reactor system containing first catalyst to produce a first reaction effluent,
withdrawing at least one side flow from a distillation column system,
supplying the at least one side flow to a second etherification reactor system containing second catalyst to produce a second reaction effluent,
mixing the second reaction effluent to the first reaction effluent,
supplying the mixture of the first and second reaction effluents to a feed point of a distillation column of the distillation column system, and
taking out the produced ether from an outlet of the above-mentioned distillation column of the distillation column system.

The above-mentioned side flow is a vapor-phase side flow, and the method according to the invention comprises condensing the vapor-phase side flow into liquid prior to supplying the side flow to the second etherification reactor system.

The etherification reactions which take place in the above-mentioned first and second etherification reactor systems are equilibrium reactions, and typically a reaction rate slows down when ether concentration increases in a reactor under consideration. The above-mentioned side flow can be arranged to be substantially free from ethers or at least a relative concentration of ethers in the side flow can be arranged to be low. Thus, the input feed to the second etherification reactor system can be substantially free from ethers and thereby the second etherification reactor system can operate in an efficient way. Therefore, thanks to the above-described side flow recycle arrangement, more ether can be produced with a same amount of catalyst material or, alternatively, a same amount of ether can be produced with a smaller amount of catalyst material.

In accordance with the invention, there is also provided a new system for producing ether. A system according to the invention comprises:
a processing system configured to receive alcohol and olefinic hydrocarbon feedstock and comprising a first etherification reactor system containing first catalyst to produce a first reaction effluent,
a distillation column system comprising one or more distillation columns, a first one of the distillation columns comprising an outlet configured to remove the produced ether from the bottom of the first distillation column,
at least one side flow outlet configured to withdraw a side flow from the distillation column system,
a second etherification reactor system containing second catalyst and configured to receive the at least one side flow and to produce a second reaction effluent, and
a piping configured to mix the second reaction effluent to the first reaction effluent and to supply the mixture of the first and second reaction effluents to a feed-point of the first distillation column.

The side flow outlet is configured to withdraw the side flow from the distillation column system in vapor-phase, and the system comprises a heat exchanger system configured to condense the vapor-phase side flow into liquid prior to supplying the side flow to the second etherification reactor system.

Exemplifying and non-limiting embodiments of the invention are described in accompanied dependent claims.

Various exemplifying and non-limiting embodiments of the invention both to constructions and to methods of operation, together with additional objects and advantages thereof, will be best understood from the following description of specific exemplifying embodiments when read in connection with the accompanying drawings.

The verbs “to comprise” and “to include” are used in this document as open limitations that neither exclude nor require the existence of also un-recited features.

The features recited in the accompanied dependent claims are mutually freely combinable unless otherwise explicitly stated.

Furthermore, it is to be understood that the use of “a” or “an”, i.e. a singular form, throughout this document does as such not exclude a plurality.

### BRIEF DESCRIPTION OF THE FIGURES

Exemplifying and non-limiting embodiments of the invention and their advantages are explained in greater details below in the sense of examples and with reference to the accompanying drawings, in which:

FIG. 1 illustrates a system according to an exemplifying and non-limiting embodiment for producing ether,

FIG. 2 illustrates a system according to another exemplifying and non-limiting embodiment for producing ether,

FIG. 3 illustrates a system not constituting a part of the invention but being used as a benchmark for illustrating an advantage of a side flow recycle arrangement illustrated in FIGS. 1 and 2, and

FIG. 4 shows a flowchart of a method according to an exemplifying and non-limiting embodiment for producing ether.

### DESCRIPTION OF EXEMPLIFYING EMBODIMENTS

The specific examples provided in the description below should not be construed as limiting the scope and/or the applicability of the accompanied claims. Lists and groups of examples provided in the description are not exhaustive unless otherwise explicitly stated.

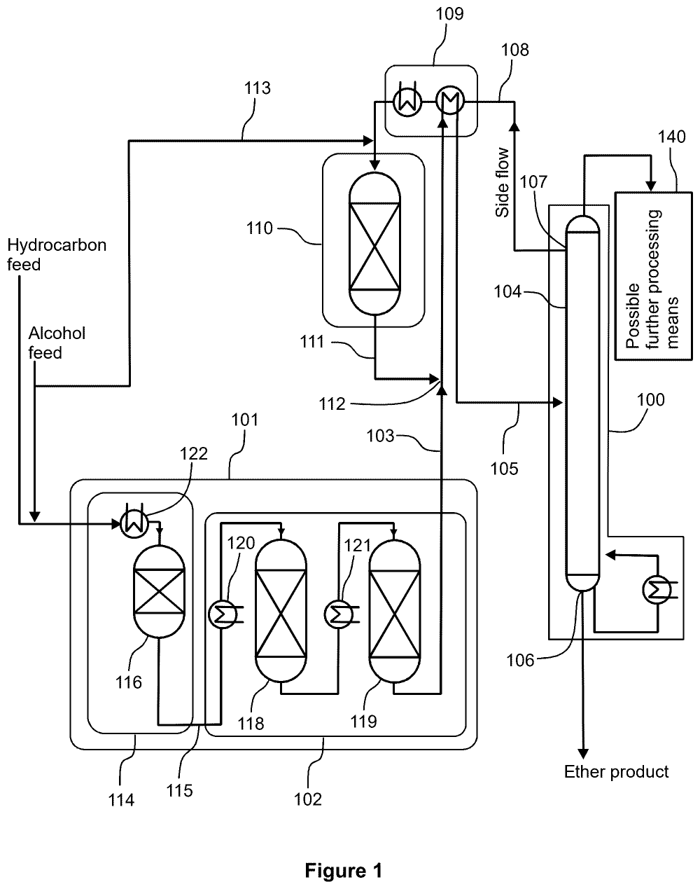

FIG. 1 illustrates a system according to an exemplifying and non-limiting embodiment for producing ether such as tertiary alkyl ether which may comprise for example methyl t-butyl ether “MTBE”, ethyl t-butyl ether “ETBE”, t-amyl methyl ether “TAME”, t-amyl ethyl ether “TAEE”, and t-hexyl methyl ether “THME”, t-hexyl ethyl ether “THEE”, and/or mixtures thereof. The exemplifying system illustrated in FIG. 1 comprises a processing system 101 configured to receive alcohol and olefinic hydrocarbon feedstock. The olefinic hydrocarbon feedstock may comprise for example C₄₋₇ olefins comprising one or more of following compounds: isobutene, 2-methyl-1-butene, 2-methyl-2-butene, 2-methyl-1-pentene, 2-methyl-2-pentene, 2,3-dimethyl-1-butene, 2,3-dimethyl-2-butene, 2-ethyl-1-butene, 2-methyl-2-hexene, 2,3-dimethyl-1-pentene, 2,3-dimethyl-2-pentene, cis-3-methyl-2-pentene, trans-3-methyl-2-pentene, 2,4-dimethyl-1-pentene, 2-ethyl-1-pentene, 1-methyl cyclopentene, and 2-ethyl-2-pentene. The processing system 101 comprises a first etherification reactor system 102 containing first catalyst in many reaction zones and configured to produce a first reaction effluent 103. The first catalyst May comprise for example cation exchange resin or zeolite. In this exemplifying case, the first etherification reactor system 102 comprises two etherification reactors 118 and 119 connected in series.

The system comprises a distillation column system 100. In this exemplifying case, the distillation column system 100 comprises one distillation column 104 that is configured to receive a mixture 105 containing the first reaction effluent 103 at a feed-point that is between the bottom and the top of the distillation column 104. The distillation column 104 comprises an outlet 106 configured to remove the produced ether from the bottom portion of the distillation column 104. The distillation column 104 comprises a side flow outlet 107 between the feed-point of the distillation column 104 and the top of the distillation column 104. The side flow outlet 107 is configured to withdraw a side flow 108 from the distillation column 104. The side flow 108 is advantageously in vapor-phase. The system comprises advantageously a heat exchanger system 109 configured to control temperature of the side flow 108. In an exemplifying case the side flow 108 is a vapor-phase side flow, the heat exchanger system 109 is advantageously configured to condense the vapor-phase side flow into liquid. The system comprises a second etherification reactor system 110 containing second catalyst in a reaction zone and configured to receive the side flow 108 and to produce a second reaction effluent 111. The second catalyst may comprise for example cation exchange resin or zeolite. The system comprises a piping 112 configured to mix the second reaction effluent 111 to the first reaction effluent 103 and to supply the mixture 105 of the first and second reaction effluents to the feed-point of the distillation column 104. The system may further comprise an inlet piping 113 configured to supply a part of the alcohol feed to the second etherification reactor system 110 to improve the ether conversion in the second etherification reactor system 110. Each of the reactors shown in FIG. 1 can be for example a fixed bed reactor, a fluidized bed reactor, an ebullated bed reactor, a boiling-point reactor, or a tubular reactor. In a fixed bed reactor, the reaction zone is a catalyst bed. In a fluidized bed reactor, the reaction zone is a fluidized catalyst bed, in an ebullated bed reactor, the reaction zone is an ebullated catalyst bed, in a boiling-point reactor the reaction zone is a catalyst bed, and, in a tubular reactor, the reaction zone is formed by parallel tubes each containing catalyst material and each constituting a sub-zone.

The etherification reactions which take place in the above-mentioned first and second etherification reactor systems 102 and 110 are equilibrium reactions, and typically a reaction rate slows down when ether concentration increases in a reactor under consideration. The side flow outlet 107 is at a such point in the distillation column 104 that the side flow 108 is substantially free from ethers or at least a relative concentration of ethers in the side flow 108 is low. Thus, the input feed to the second etherification reactor system 110 is substantially free from ethers and thereby the second etherification reactor system 110 provides an efficient ether production for a given amount of catalyst material. Thus, thanks to the side flow recycle arrangement, more ether can be produced with a same amount of catalyst material or, alternatively, a same amount of ether can be produced with a smaller amount of catalyst material.

In the exemplifying system illustrated in FIG. 1, the heat exchanger system 109 is configured to transfer heat from the side flow 108 to the mixture 105 of the first and second reaction effluents prior to supplying the mixture 105 to the distillation column 104. This reduces the energy consumption of the system for producing ether.

In the exemplifying system illustrated in FIG. 1, the processing system 101 comprises a third etherification reactor system 114 configured to receive the alcohol and the olefinic hydrocarbon feedstock. The third etherification reactor system 114 contains third catalyst in a reaction zone to produce a third reaction effluent 115 that is supplied to the first etherification reactor system 102. The third catalyst may comprise for example cation exchange resin or zeolite. In this exemplifying system, the third etherification reactor system 114 comprises an etherification reactor 116. The etherification reactor 116 acts as a guard-bed reactor which removes unwanted components from the olefinic hydrocarbon feedstock and from the alcohol supplied to the third etherification reactor system 114. For example, fluid catalytic cracking “FCC”-derived olefinic liquefied petroleum gas “LPG” may contain basic nitrogen compounds which are direct catalyst poisons, as well as other basic material, ionic metal compounds and other deactivators. Therefore, the third etherification reactor system 114 protects the second etherification reactor system 102 from catalyst deactivation in addition to contributing the main process to produce ether. Therefore, the exemplifying system illustrated in FIG. 1 can be, and is, free from a water-wash column.

In a system according to another exemplifying and non-limiting embodiment for producing ether, the processing system 101 comprises a water-wash column configured to wash the olefinic hydrocarbon feedstock prior to supplying the olefinic hydrocarbon feedstock to the first etherification reactor system 102. The system comprising the water-wash column is not shown in the figures.

The catalyst material of the third etherification reactor system 114 needs to be changed more often than the catalyst material of the first etherification reactor system 102 and the catalyst material of the second etherification reactor system 110, but equipment costs of the third etherification reactor system 114, material costs of the catalyst material to be changed, and labor costs for changing the catalyst material of the third etherification reactor system 114 are smaller than for example corresponding capital and operating costs related to a water-wash column. The volume of the third catalyst contained by the etherification reactor 116 is at most 15% of the total volume of the third catalyst and the first catalyst that is contained by the etherification reactors 118 and 119 of the first etherification reactor system 102. In other words, the volume of the third catalyst within the reactor 116 is at most about 18% of the volume of the first catalyst contained by the reactors 118 and 119. Advantageously, the volume of the third catalyst is from 10% to 15% of the total volume of the first and third catalyst.

The first etherification reactor system 102 further comprises heat exchangers 120 and 121 for controlling temperatures of materials supplied to the etherification reactors 118 and 119. Correspondingly, the third etherification reactor system 114 comprises a heat exchanger 122 for controlling temperature of materials supplied to the etherification reactor 116 that acts as the guard-bed reactor. In FIG. 1, temperature control means connected to the heat exchangers 120-122 are not shown. The input temperatures of the etherification reactors are typically in the range from 30° C. to 60° C., more advantageously from 40° C. to 45° C., and the output temperatures of the etherification reactors are advantageously at most from 70° C. to 80° C. The pressure within each etherification reactor is advantageously set to a value so that evaporation of C₄ hydrocarbons can either be avoided or controlled to maintain required temperature level in the reactor in case a boiling point reactor configuration is employed. The pressure within each etherification reactor is advantageously in the range from 1700 kPa to 1900 kPa.

The system may further comprise means 140 for further processing a material flow drawn out from the upper portion of the distillation column 104. The means 140 may comprise for example another distillation column whose operating pressure is higher than that of the distillation column 104. The operating pressure in the distillation column 104 can be typically in the range from 600 to 900 kPa, and in practice less than 1000 kPa. The operating pressure in the other distillation column can be typically in the range from 1500 to 2400 kPa, and in practice less than 3000 kPa. The means 140 may further comprise side flow feed-back arrangements from the other distillation column to one or more suitable points in the first etherification reactor system 102 and/or in the third etherification reactor system 114. For example, a side flow feed-back arrangement can be configured to draw a side flow from an upper part of the other distillation column and to supply the side flow to the inlet of the third etherification reactor system 114.

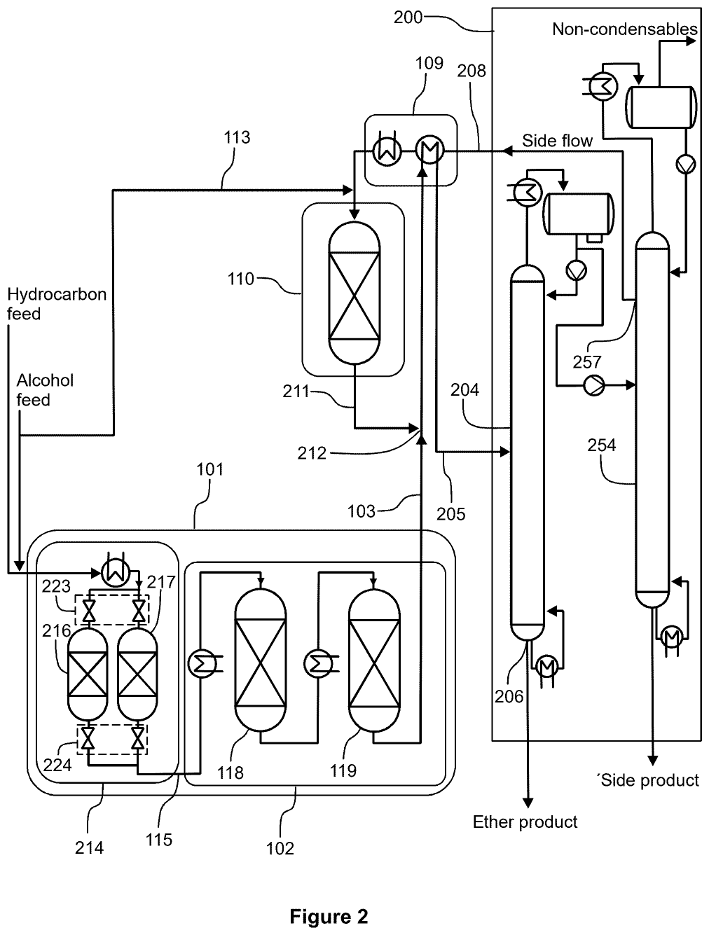

FIG. 2 illustrates a system according to an exemplifying and non-limiting embodiment for producing ether such as tertiary alkyl ether or a mixture of tertiary alkyl ethers. In the exemplifying system illustrated in FIG. 2, a distillation column system 200 comprises a first distillation column 204 that is configured to receive a mixture 205 containing the first reaction effluent 103 and a second reaction effluent 211 at a feed-point that is between the bottom and the top of the first distillation column 204. The first distillation column 204 comprises an outlet 206 configured to remove the produced ether from the bottom portion of the first distillation column 204. The distillation column system 200 comprises a second distillation column 254 that is configured to receive a flow from the first distillation column 204 at a feed-point that is between the bottom and the top of the second distillation column 254. The second distillation column comprises the side flow outlet 257 between the feed-point of the second distillation column 254 and the top of the second distillation column 254. The side flow outlet 257 is configured to withdraw a side flow 208 from the distillation column 254. The side flow 208 is advantageously in vapor-phase.

The system comprises advantageously a heat exchanger system 109 configured to control temperature of the side flow 208. In an exemplifying case where the side flow 208 is a vapor-phase side flow, the heat exchanger system 109 is advantageously configured to condense the vapor-phase side flow into liquid prior to supplying the side flow 208 to the second etherification reactor system 110. The operating pressure in the first distillation column 204 can be typically in the range from 600 to 900 kPa, and in practice less than 1000 kPa. The operating pressure in the second distillation column 254 can be typically in the range from 1500 to 2400 kPa, and in practice less than 3000 kPa.

In the exemplifying system illustrated in FIG. 2, the third etherification reactor system 214 comprises two etherification reactors 216 and 217 connected in parallel and valves 223 and 224 for disconnecting each of the etherification reactors 216 and 217 from the other reactor of the third etherification reactor system 214. The valves 223 and 224 enable each of the etherification reactors 216 and 217 to be disconnected in response to a need to replace deactivated catalyst material of the reactor under consideration with active catalyst material and to reconnect the disconnected reactor into operation, or to be ready for operation, after replacing its catalyst material. Catalyst materials of the etherification reactors 216 and 217 can be changed in an alternating way so that one of the etherification reactors 216 and 217 is in use in the main process for producing ether when catalyst material of the other one of the reactors 216 and 217 is being changed. Thus, there is no need to stop the main process during changing the catalyst material of the third etherification reactor system 214. The volume of the catalyst that contributes the production of the third reaction effluent 115 within the third etherification reactor system 214 is at most 15% of the total volume of the catalyst contributing the production of the first reaction effluent 103 within the first etherification reactor system 102 and the catalyst contributing the production of the third reaction effluent 115. Thus, in a case where only one of the reactors 216 and 217 is used at a time, the volume of the catalyst within each of the reactors 216 and 217 is the above-mentioned at most 15% of the above-mentioned total volume. Correspondingly, in a case where both the reactors 216 and 217 are used simultaneously, the volume of the catalyst within each of the reactors 216 and 217 is at most 7.5% of the above-mentioned total volume. Each of the reactors shown in FIG. 2 can be for example a fixed bed reactor, a fluidized bed reactor, an ebullated bed reactor, a boiling-point reactor, or a tubular reactor.

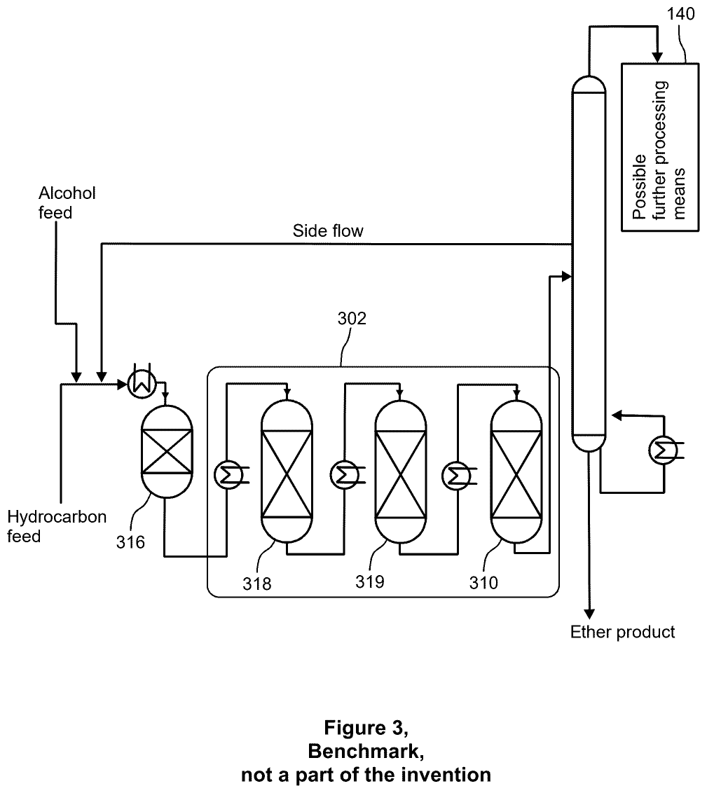

FIG. 3 illustrates a system that does not constitute a part of the invention, but the system shown in FIG. 3 is used as a benchmark for illustrating the advantage of the side flow recycle arrangement illustrated in FIGS. 1 and 2. The system shown in FIG. 3 is otherwise like the system shown in FIG. 1, but the system shown in FIG. 3 does not comprise the side flow recycle arrangement shown in FIG. 1, the first etherification reactor system 302 of the system shown in FIG. 3 comprises three etherification reactors 318, 319, and 310 connected in series, and the system shown in FIG. 3 comprises a side flow recycle arrangement connected to the inlet of an etherification reactor 316 that acts as a guard-bed reactor. Each of the reactors shown in FIG. 3 can be for example a fixed bed reactor, a fluidized bed reactor, an ebullated bed reactor, a boiling-point reactor, or a tubular reactor.

An operational example of a system of the kind described above with reference to FIG. 1 is presented below. In this example case, the system is configured to produce ethyl t-butyl ether “ETBE”.

With the aid of simulations, it was found that the side flow 108 drawn from the distillation column 104 and returned to the inlet of the second etherification reactor system 110 has a beneficial impact on the amount of catalyst needed. In other words, the amount of catalyst can be reduced for a given amount of ETBE product or a greater amount of ETBE product can be produced with a given amount of catalyst. In this example, the side flow 108 is a vapor-phase side flow.

Table 1 that is presented below shows a comparison between a system according to FIG. 1 for ethyl t-butyl ether “ETBE” production and a system according to FIG. 3 in which a side flow is drawn from the distillation column and returned to the inlet of the etherification reactor 316.

As can be seen by comparing the columns of Table 1 related to the system according to FIG. 1 and to the system according to FIG. 3, the system according to FIG. 1 provides substantially the same performance but with 45 m³ smaller total volume of the catalyst.

TABLE 1
Comparison of overall results for the system
of FIG. 1 and the benchmark system of FIG. 3.

| | Benchmark | |
|---|---|---|
| | System | System |
| | according to | according to |
| Entity | FIG. 3 | FIG. 1 |
| Outlet temperature ° C. of the reactor 118 | 68.8 | 68.8 |
| in FIG. 1 and the reactor 318 in FIG. 3 | | |
| Outlet temperature ° C. of the reactor 119 | 61.3 | 61.3 |
| in FIG. 1 and the reactor 319 in FIG. 3 | | |
| Outlet temperature ° C. of the reactor of | 41.5 | 44.9 |
| the etherification reactor system 110 in | | |
| FIG. 1 and the reactor 310 in FIG. 3 | | |
| Catalyst volume m³ the reactor 118 in | 36 | 36 |
| FIG. 1 and the reactor 318 in FIG. 3 | | |
| Catalyst volume m³ of the reactor 119 in | 90 | 90 |
| FIG. 1 and the reactor 319 in FIG. 3 | | |
| Catalyst volume m³ of the reactor of the | 90 | 45 |
| etherification reactor system 110 in | | |
| FIG. 1 and the reactor 310 in FIG. 3 | | |
| Overall iso-butene conversion, % | 98.0 | 98.0 |
| Product ETBE flow, kg/h | 39225 | 39225 |
| Product ETBE content, % | 98.1 | 98.1 |
| Product ethanol content, % | 0.98 | 0.98 |
| Product water content, % | 0.03 | 0.03 |
| Product TBA content, % | 0.85 | 0.86 |
| Product DEE content, % | 0.02 | 0.009 |
| Product C₄ content, % | 0.000 | 0.000 |

The iso-butene conversion ratio, the product ETBE content weight-%, and the product ethanol content weight-% of the system according to FIG. 1 can be optimized by selecting a location of the side flow outlet 107 in the vertical direction of the distillation column 104 and by selecting a flow rate, kg/h, of the side flow 108 in an advantageous way.

The functionality of a guard-bed reactor for removing impurities is compared to functionality of a water-wash column in operational examples illustrated with the tables shown below. In a first example case, a system according to FIG. 3, which comprises the guard-bed reactor 316, is configured to produce ethyl t-butyl ether “ETBE”:

TABLE 2
Hydrocarbon feed 18150.6 kg/h to the system comprising
the guard-bed reactor, the reactor 316 in FIG. 3

| Composition: | kmol/h | kg/h |
|---|---|---|
| Propene | 0.043 | 1.8 |
| Propane | 0.84 | 36.9 |
| 1,3-Butadiene | 1.18 | 63.5 |
| Isobutene | 161.2 | 9042.9 |
| Linear butenes | 72.71 | 4079.4 |
| Butanes | 84.74 | 4925.1 |
| Isopentane | 0.001 | 0.1 |
| Acetonitrile | 0.022 | 0.9 |

TABLE 3
Ethanol feed 7672.5 kg/h to the system comprising
the guard-bed reactor, the reactor 316 in FIG. 3

| Composition: | kmol/h | kg/h |
|---|---|---|
| H₂O | 0.426 | 7.7 |
| MEOH | 0.191 | 6.1 |
| ETOH | 165.41 | 7620.3 |
| C₅ alcohol | 0.435 | 38.4 |

TABLE 4
Reactor sizes, i.e. volumes of catalyst in the system comprising
the guard-bed reactor, the reactor 316 in FIG. 3

| | |
|---|---|
| Reactor 316: | 8 m³ |
| Reactor 318: | 12 m³ |
| Reactor 319: | 25 m³ |
| Reactor 310: | 25 m³ |
| Total: | 70 m³ |

Total conversion of isobutene in the system comprising the guard-bed reactor: 97.65%

TABLE 5
ETBE product 16502 kg/h in the system comprising
the guard-bed reactor, the reactor 316 in FIG. 3

| Composition: | kmol/h | kg/h |
|---|---|---|
| 1,3-Butadiene | 0.0002 | 0.006 |
| Isobutene | 0.0004 | 0.03 |
| Linear butenes | 0.0092 | 0.51 |
| Butanes | 0.0047 | 0.28 |
| Isopentane | 0.0012 | 0.09 |
| 2,4,4-trimethylpentene | 0.0004 | 0.05 |
| H₂O | 0.0601 | 1.1 |
| Tert-butyl alcohol | 0.4079 | 30.2 |
| MEOH | 0.00006 | 0.0 |
| MTBE | 0.1809 | 16.0 |
| ETOH | 8.4921 | 391.2 |
| Diethyl-ether | 0.0583 | 4.3 |
| Acetonitrile | 0.0 | 0.0 |
| C₅ alcohol | 0.4352 | 38.4 |
| ETBE | 156.7889 | 16020.1 |

A corresponding operational example of a system which is otherwise like the system related to the above-presented tables 2-5 but which comprises a water-wash column instead of a guard-bed reactor is presented with the tables below. The main purpose of the water-wash column is to remove acetonitrile from the hydrocarbon feed. In this operational example, the diameter of the water-wash column is 1.2 m, the height of the water-wash column is 23 m, and the water-wash column has 40 trays. In a case of 50 weight-ppm acetonitrile concentration in hydrocarbon feed having a flow rate of 18150 kg/h, a need for washing water is about 11000 kg/h to achieve a target level 1 weight-ppm in the acetonitrile concentration. The operational temperature of the water-wash column can be for example in the range from 15° C. to 50° C., more advantageously from 20° C. to 40° C., and the operational pressure of the water-wash column can be in the range from 800 kPa to 1000 kPa.

In the system comprising the water-wash column, the etherification reactor system comprises three reactors which are connected in series and whose catalyst volumes are:

| | |
|---|---|
| The first reactor after the water-wash column | 20 m³ |
| The second reactor after the water-wash column: | 25 m³ |
| The third reactor after the water-wash column: | 25 m³ |
| Total: | 70 m³. |

The first reactor after the water-wash column is provided with a feedback circulation around the first reactor to limit temperature in the first reactor so that the output temperature of the first reactor does not exceed 70° C. to avoid a decrease in selectivity to ETBE at higher temperatures. In the system comprising the guard-bed reactor, there is no need for a feedback circulation of the kind mentioned above.

TABLE 6
Hydrocarbon feed 18150.6 kg/h to the system
comprising the water-wash column

| Composition: | kmol/h | kg/h |
|---|---|---|
| Propene | 0.043 | 1.8 |
| Propane | 0.84 | 36.9 |
| 1,3-Butadiene | 1.18 | 63.5 |
| Isobutene | 161.2 | 9042.9 |
| Linear butenes | 72.71 | 4079.4 |
| Butanes | 84.74 | 4925.1 |
| Isopentane | 0.001 | 0.1 |
| Acetonitrile | 0.022 | 0.9 |

TABLE 7
Ethanol feed 7669.8 kg/h to the system
comprising the water-wash column

| Composition: | kmol/h | kg/h |
|---|---|---|
| H₂O | 0.426 | 7.7 |
| MEOH | 0.192 | 6.1 |
| ETOH | 165.35 | 7617.6 |
| C₅ alcohol | 0.435 | 38.4 |

Total conversion of isobutene in the system comprising the water-wash column: 97.64%

TABLE 8
ETBE product 16500 kg/h in the system
comprising the water-wash column

| Composition: | kmol/h | kg/h |
|---|---|---|
| 1,3-Butadiene | 0.0001 | 0.006 |
| Isobutene | 0.0003 | 0.02 |
| Linear butenes | 0.0063 | 0.35 |
| Butanes | 0.0036 | 0.21 |
| Isopentane | 0.0012 | 0.09 |
| 2,4,4-trimethylpentene | 0.0005 | 0.06 |
| H₂O | 0.096 | 1.7 |
| Tert-butyl alcohol | 1.0024 | 40.9 |
| MEOH | 0.00006 | 0.0 |
| MTBE | 0.18 | 15.9 |
| ETOH | 8.60 | 396.3 |
| Diethyl-ether | 0.063 | 4.7 |
| Acetonitrile | 0.0 | 0.0 |
| C₅ alcohol | 0.44 | 38.4 |
| ETBE | 156.61 | 16001.6 |

As can be seen by comparing the above-presented examples, the system comprising the guard-bed reactor provides substantially the same performance with the same total volume 70 m³ of the catalyst material but without a need for the water-wash column whose capital and operating costs are higher than those of the guard-bed reactor. Furthermore, the water washing produces wastewater about 11000 kg/h.

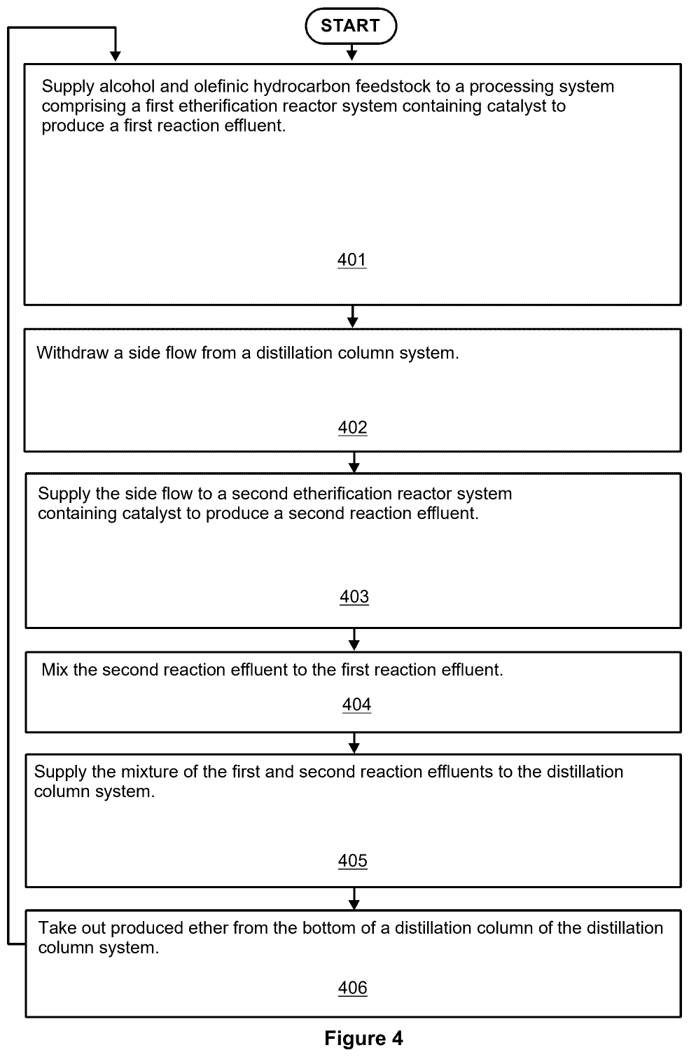

FIG. 4 shows a flowchart of a method according to an exemplifying and non-limiting embodiment for producing ether such as tertiary alkyl ether or a mixture of tertiary alkyl ethers. The method comprises the following actions:

action 401: supplying alcohol and olefinic hydrocarbon feedstock, for example C₄₋₇ olefins, to a processing system comprising a first etherification reactor system containing first catalyst in one or more reaction zones to produce a first reaction effluent,
action 402: withdrawing at least one side flow from a distillation column system, action 403: supplying the at least one side flow to a second etherification reactor system containing second catalyst in one or more reaction zones to produce a second reaction effluent,
action 404: mixing the second reaction effluent to the first reaction effluent,
action 405: supplying the mixture of the first and second reaction effluents to a feed point of a distillation column of the distillation column system, and
action 406: taking out the produced ether from an outlet of the above-mentioned distillation column of the distillation column system.

The above-described method is advantageously a continuous process, which is illustrated by the arrow from the action 406 back to the action 401.

The above-mentioned side flow can be arranged to be substantially free from ethers or at least a relative concentration of ethers in the side flow can be arranged to be low. Thus, the input feed to the second etherification reactor system can be substantially free from ethers and thereby the second etherification reactor system can operate in an efficient way. Therefore, thanks to the side flow recycle arrangement, more ether can be produced with a same amount of catalyst material or, alternatively, a same amount of ether can be produced with a smaller amount of catalyst material.

In a method according to an exemplifying and non-limiting embodiment, the above-mentioned side flow is withdrawn from a side flow outlet of the above-mentioned distillation column, where the side flow outlet is between the feed-point of the distillation column and the top of the distillation column.

In a method according to an exemplifying and non-limiting embodiment, the distillation column is a first distillation column of the distillation column system, the distillation column system comprises a second distillation column receiving a flow from the first distillation column at a feed-point of the second distillation column, and the side flow is withdrawn from a side flow outlet of the second distillation column between the feed-point of the second distillation column and the top of the second distillation column.

In a method according to an exemplifying and non-limiting embodiment, the above-mentioned side flow is a vapor-phase side flow, and the method comprises condensing the vapor-phase side flow into liquid prior to supplying the side flow to the second etherification reactor system.

In a method according to an exemplifying and non-limiting embodiment, alcohol is supplied to the second etherification reactor system in addition to the side flow.

A method according to an exemplifying and non-limiting embodiment comprises cooling down the above-mentioned side flow and warming up the mixture of the first and second reaction effluents to be supplied to the distillation column system by transferring heat from the side flow to the mixture of the first and second reaction effluents with a heat-exchanger.

In a method according to an exemplifying and non-limiting embodiment, the first catalyst in the first etherification reactor system comprises cation exchange resin or zeolite, and the second catalyst in the second etherification reactor system comprises cation exchange resin or zeolite. In a method according to an exemplifying and non-limiting embodiment, the first catalyst is the same material as the second catalyst.

In a method according to an exemplifying and non-limiting embodiment, the processing system comprises a third etherification reactor system containing third catalyst in one or more reaction zones, and the alcohol and the olefinic hydrocarbon feedstock are supplied to the third etherification reactor system to produce a third reaction effluent which is supplied to the first etherification reactor system. The third catalyst may comprise for example cation exchange resin or zeolite. In a method according to an exemplifying and non-limiting embodiment, the first, second, and third catalysts are the same catalyst material.

The volume of the above-mentioned third catalyst contributing the production of the third reaction effluent within the third etherification reactor system is at most 15% of the total volume of the third catalyst contributing the production of the third reaction effluent and the first catalyst contributing the production of the first reaction effluent within the first etherification reactor system. The method according to this exemplifying and non-limiting embodiment comprises replacing the deactivated third catalyst of the third etherification reactor system with active catalyst material more often than replacing the deactivated first catalyst of the first etherification reactor system with active catalyst material. The third etherification reactor system acts as a guard-bed reactor which removes unwanted components from the feed materials supplied to the first etherification reactor system. Thus, the third etherification reactor system protects the first etherification reactor system from catalyst deactivation in addition to contributing the main process to produce ether. Therefore, catalyst of the third etherification reactor system needs to be changed more often than that of the first etherification reactor system. In a method according to another exemplifying and non-limiting embodiment, the processing system comprises a water-wash column configured to wash the olefinic hydrocarbon feedstock to remove unwanted substances from the olefinic hydrocarbon feedstock.

In a method according to an exemplifying and non-limiting embodiment, the above-mentioned volume of the third catalyst is from 10% to 15% of the above-mentioned total volume of the first and third catalysts.

In a method according to an exemplifying and non-limiting embodiment, the third etherification reactor system comprises at least two reactors connected in parallel and valves for disconnecting each of the reactors from the one or more other reactors of the third etherification reactor system, and the method comprises:
disconnecting, with the valves, one of the reactors of the third etherification reactor system in response to a situation which the third catalyst of the one of the reactors has been deactivated,
replacing the third catalyst of the disconnected reactor with active catalyst material, and
reconnecting the disconnected reactor into operation after replacing its third catalyst.

A method according to an exemplifying and non-limiting embodiment comprises changing the third catalyst of the above-mentioned at least two reactors of the third etherification reactor system in an alternating way in accordance with a predetermined timing schedule.

In a method according to an exemplifying and non-limiting embodiment, the first etherification reactor system comprises two or more etherification reactors connected in series.

In a method according to an exemplifying and non-limiting embodiment, the olefinic hydrocarbon feedstock comprises C₄₋₇ olefins comprising one or more of following: isobutene, 2-methyl-1-butene, 2-methyl-2-butene, 2-methyl-1-pentene, 2-methyl-2-pentene, 2,3-dimethyl-1-butene, 2,3-dimethyl-2-butene, 2-ethyl-1-butene, 2-methyl-2-hexene, 2,3-dimethyl-1-pentene, cis-3-methyl-2-pentene, trans-3-methyl-2-pentene, 2,3-dimethyl-2-pentene, 2,4-dimethyl-1-pentene, 2-ethyl-1-pentene, 1-methyl cyclopentene, and 2-ethyl-2-pentene.

The specific examples provided in the description given above should not be construed as limiting. Therefore, the invention is not limited merely to the exemplifying and non-limiting embodiments described above. Lists and groups of examples provided in the description are not exhaustive unless otherwise explicitly stated.

1. A method for producing ether, the method comprising:
supplying alcohol and olefinic hydrocarbon feedstock to a processing system including a first etherification reactor system containing a first catalyst to produce a first reaction effluent;
supplying a mixture containing the first reaction effluent to a feed-point of a distillation column of a distillation column system;
taking out a produced ether from an outlet of the distillation column of the distillation column system;
withdrawing at least one side flow from the distillation column system;
supplying the at least one side flow to a second etherification reactor system containing a second catalyst to produce a second reaction effluent; and
mixing the second reaction effluent to the first reaction effluent prior to supplying a mixture of the first and second reaction effluents to the distillation column of the distillation column system;
wherein the side flow is a vapor-phase side flow, and the method includes condensing the vapor-phase side flow into liquid prior to supplying the at least one side flow to the second etherification reactor system.

2. A method according to claim 1, composing;
withdrawing the at least one side flow from a side flow outlet of the distillation column of the distillation column system, the side flow outlet being between the feed-point of the distillation column and a top of the distillation column.

3. A method according to claim 1, wherein the distillation column is a first distillation column of the distillation column system, and the distillation column system includes a second distillation column, the method comprising:
receiving a flow from the first distillation column at a feed-point of the second distillation column; and
withdrawing the side flow from a side flow outlet of the second distillation column between the feed-point of the second distillation column and a top of the second distillation column.

4. A method according to claim 1, comprising:
supplying alcohol to the second etherification reactor system in addition to the side flow.

5. A method according to claim 1, comprising:
cooling down the side flow and warming up the mixture of the first and second reaction effluents to be supplied to the distillation column system by transferring heat from the at least one side flow to the mixture of the first and second reaction effluents with a heat-exchanger.

6. A method according to claim 1, wherein the first catalyst is same material as the second catalyst.

7. A method according to claim 1, wherein the processing system includes a third etherification reactor system containing a third catalyst, the method comprising:
supplying the alcohol and the olefinic hydrocarbon feedstock to the third etherification reactor system to produce a third reaction effluent which is supplied to the first etherification reactor system, and a volume of the third catalyst contributing to a production of the third reaction effluent within the third etherification reactor system is at most 15% of a total volume of the third catalyst contributing the production of the third reaction effluent and the first catalyst contributing a production of the first reaction effluent within the first etherification reactor system; and
replacing, in response to a deactivation of the third catalyst, the deactivated third catalyst with active catalyst material more often than replacing, in response to deactivation of the first catalyst, the deactivated first catalyst with active catalyst material.

8. A method according to claim 7, wherein the volume of the third catalyst is from 10% to 15% of the total volume of the first and third catalysts.

9. A method according to claim 7, wherein the third etherification reactor system includes at least two reactors connected in parallel and valves for disconnecting each of the reactors from the one or more other reactors of the third etherification reactor system, and the method comprises:
disconnecting, with the valves, one of the reactors of the third etherification reactor system in response to a situation in which the third catalyst of the one of the reactors has been deactivated;
replacing the third catalyst of the disconnected reactor with active catalyst material; and
reconnecting the disconnected reactor into operation, or to be ready for operation, after replacing its third catalyst.

10. A method according to claim 9, comprising:
changing the third catalyst of the at least two reactors of the third etherification reactor system in an alternating way in accordance with a predetermined timing schedule.

11. A method according to claim 1 wherein the first etherification reactor system includes two or more reactors connected in series.

12. A method according to claim 1, wherein the ether is tertiary alkyl ether or a mixture of tertiary alkyl ethers.

13. A method according to claim 1, wherein the first catalyst includes cation exchange resin or zeolite, and the second catalyst includes cation exchange resin or zeolite.

14. A method according to claim 1, wherein the olefinic hydrocarbon feedstock includes C₄₋₇ olefins including at least one or more of a following: isobutene, 2-methyl-1-butene, 2-methyl-2-butene, 2-methyl-1-pentene, 2-methyl-2-pentene, 2,3-dimethyl-1-butene, 2,3-dimethyl-2-butene, 2-ethyl-1-butene, 2-methyl-2-hexene, 2,3-dimethyl-1-pentene, cis-3-methyl-2-pentene, trans-3-methyl-2-pentene, 2,3-dimethyl-2-pentene, 2,4-dimethyl-1-pentene, 2-ethyl-1-pentene, 1-methyl cyclopentene, and/or 2-ethyl-2-pentene.

15. A system for producing ether, the system comprising:
a processing system configured to receive alcohol and olefinic hydrocarbon feedstock and including a first etherification reactor system containing a first catalyst to produce a first reaction effluent; and
a distillation column system configured to receive a mixture containing the first reaction effluent, wherein the distillation column system includes:
a distillation column having at a feed-point configured to receive the mixture and an outlet configured to remove produced ether from the distillation column, and the system comprises:
at least one side flow outlet configured to withdraw at least one side flow from the distillation column system;
a second etherification reactor system containing a second catalyst and configured to receive the at least one side flow and to produce a second reaction effluent; and
a piping configured to mix the second reaction effluent to the first reaction effluent and to supply the mixture of the first and second reaction effluents to the feed-point of the distillation column,
wherein the at least one side flow outlet is configured to withdraw at least one side flow from the distillation column system as a vapor-phase side flow, and the system includes:
a heat exchanger system configured to condense the vapor-phase side flow into liquid prior to supplying the at least one side flow to the second etherification reactor system.

16. A system according to claim 15, wherein the distillation column comprises:
the at least one side flow outlet between the feed-point of the distillation column and a top of the distillation column.

17. A system according to claim 15, wherein the distillation column is a first distillation column of the distillation column system, and the distillation column system comprises:
a second distillation column configured to receive a flow from the first distillation column at a feed-point of the second distillation column, and the second distillation column includes the at least one side flow outlet between the feed-point of the second distillation column and a top of the second distillation column.

18. A system according to claim 15, comprising:
an inlet piping configured to supply alcohol to the second etherification reactor system.

19. A system according to claim 15, wherein the heat exchanger system is configured to transfer heat from the at least one side flow to the mixture of the first and second reaction effluents to be supplied to the distillation column system, and the second etherification reactor system is configured to receive the at least one side flow from the heat exchanger system.

20. A system according to claim 15, wherein the processing system comprises;
a third etherification reactor system configured to receive the alcohol and the olefinic hydrocarbon feedstock, containing a third catalyst to produce a third reaction effluent, and configured to supply the third reaction effluent to the first etherification reactor system, and wherein a volume of the third catalyst contributing to a production of the third reaction effluent within the third etherification reactor system is at most 15% of a total volume of the third catalyst contributing the production of the third reaction effluent and the first catalyst contributing a production of the first reaction effluent within the first etherification reactor system.

21. A system according to claim 20, wherein the volume of the third catalyst is from 10% to 15% of the total volume of the first and third catalysts.

22. A system according to claim 20, wherein the third etherification reactor system comprises:
at least two reactors connected in parallel, and valves for disconnecting each of the reactors from one or more other reactors of the third etherification reactor system, the valves enabling each of the reactors of the third etherification reactor system to be disconnected to replace the third catalyst of the reactor with active catalyst material and to reconnect the disconnected reactor into operation, or to be ready for operation, after replacing the third catalyst of the reactor.

23. A system according to claim 15, wherein the first etherification reactor system comprises
two or more etherification reactors connected in series.
